(19) United States
(12) Patent Application Publication
(10) Pub. No.: US 2023/0343427 A1
(43) Pub. Date: Oct. 26, 2023

(54) INFORMATION PROCESSING METHOD, TERMINAL DEVICE, AND NON-TRANSITORY COMPUTER-READABLE RECORDING MEDIUM

(71) Applicant: Panasonic Holdings Corporation, Osaka (JP)

(72) Inventors: YUKIHIRO MORITA, Osaka (JP); NAOKI SAITO, Tokyo (JP); NAOTO AGU, Tokyo (JP); TSUKASA KANEKO, Chiba (JP)

(21) Appl. No.: 18/347,618

(22) Filed: Jul. 6, 2023

Related U.S. Application Data
(63) Continuation of application No. PCT/JP2022/000739, filed on Jan. 12, 2022.

(30) Foreign Application Priority Data
Jan. 29, 2021 (JP) 2021-013779

(51) Int. Cl. G16H 15/00; G16H 50/30; G16H 10/20

(52) U.S. Cl. CPC G16H 15/00; G16H 50/30; G16H 10/20; G16H 20/70

(57) ABSTRACT

This information processing method is to be performed by a computer and includes acquiring n types (where n is an integer equal to or greater than 1) of physiological measures of a person in a state for meditation, executing determination processing that determines whether each of the acquired n types of physiological measures exceeds a threshold value corresponding to that type of physiological measure; executing derivation processing that derives a score related to a meditative state of the person on the basis of the result of the determination processing, and outputting the derived score.

### BACKGROUND

### 1. Technical Field

The present disclosure relates to an information processing method and the like related to meditation.

### 2. Description of the Related Art

In the related art, a meditation guidance device that guides a user into a meditative state to steady the autonomic nervous system has been proposed (for example, see Japanese Unexamined Patent Application Publication No. 2020-58470, hereinafter referred to as JP 2020-58470A). In this meditation guidance device, the wavelength of light emitted from a light emitter toward a user with eyes closed is corrected according to the light transmission characteristics of the eyelids so that the emitted light appears to be in the greenish to yellowish wavelength range. Additionally, the meditation guidance device changes the emission state of the emitted light to inform the user of information related to meditation guidance. With this arrangement, the user with eyes closed can be guided into a meditative state to steady the autonomic nervous system without adversely affecting the user.

### SUMMARY

However, an information processing method by the meditation guidance device according to JP 2020-58470A has a problem in that it is difficult to motivate the user to meditate repeatedly in an ongoing way.

One non-limiting and exemplary embodiment provides an information processing method that can motivate a person to meditate and keep that person meditating repeatedly in an ongoing way.

In one general aspect, the techniques disclosed here feature an information processing method which is to be performed by a computer and which includes: executing acquisition processing that acquires n types (where n is an integer equal to or greater than 1) of physiological measures of a person in a state for meditation; executing determination processing that determines whether each of the acquired n types of physiological measures exceeds a threshold value corresponding to that type of physiological measure; executing derivation processing that derives a score related to a meditative state of the person on the basis of the result of the determination processing; and outputting the derived score.

The information processing method according to the present disclosure can motivate a person to meditate and keep that person meditating repeatedly in an ongoing way.

It should be noted that general or specific embodiments may be implemented as an apparatus, a system, an integrated circuit, a computer program, a computer-readable recording medium, or any selective combination thereof. The computer-readable recording medium includes a non-volatile recording medium such as Compact Disc-Read-Only Memory (CD-ROM), for example.

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1 is a diagram illustrating an experimental protocol;

FIG. 2 is a diagram illustrating P-values of physiological measures obtained through experiment;

FIG. 3 is a block diagram illustrating a configuration of an information processing system according to an embodiment;

FIG. 4A is a diagram illustrating an electrocardio waveform for explaining heartbeat interval in an embodiment;

FIG. 4B is a diagram for explaining heart rate variability in the embodiment;

FIG. 4C is a diagram for explaining respiratory cycle in the embodiment;

FIG. 5 is a diagram for explaining the derivation of a score in the embodiment;

FIG. 6 is a flowchart illustrating an example of general score-related processing operations by a terminal device in the embodiment;

FIG. 7 is a diagram for explaining threshold parameters of physiological measures in the embodiment;

FIG. 8 is a diagram for explaining threshold parameters of other physiological measures in the embodiment;

FIG. 9 is a diagram illustrating an example of screen transitions on a terminal device in the embodiment;

FIG. 10 is a diagram illustrating an example of a questionnaire screen in the embodiment;

FIG. 11 is a diagram illustrating an example of a results display screen in the embodiment; and

FIG. 12 is a flowchart illustrating an example of processing operations by the terminal device in the embodiment.

### DETAILED DESCRIPTIONS

### (Underlying Knowledge Forming Basis of the Present Disclosure)

The inventors discovered the following problem in relation to the information processing method by the meditation guidance device of JP 2020-58470A cited in the “Background Art” section above.

In the information processing method of JP 2020-58470A, there is no feedback of meditation results indicating, among other things, how well a person meditated. Consequently, there is little sense of accomplishment for a person who experiences meditation. For this reason, there is reduced motivation to continue meditating. It is said that the effects of meditation are often difficult to achieve after only a few meditation sessions, and it is important to continue meditating to improve one's ability to meditate.

Accordingly, the inventors conducted experiments on subjects and thereby arrived at a method of using physiological measures to score and provide feedback regarding a meditative state.

In the experiments, multiple types of physiological measures were obtained from each of experienced and novice meditators. For the experienced meditators, subjects with at least 1500 hours of past meditation time were recruited, and 151 persons responded. Of these 151 persons, 17 persons were selected as experienced subjects. The 17 persons were men and women in their 20s to 60s, and the average meditation time for the 17 persons was 4931 hours. For the novice meditators, 28 men and women in their 20s to 50s with little or no meditation experience were selected as novice subjects.

The types of physiological measures are the heartbeat interval and heart rate variability obtained by measurement of an electrocardio waveform, and the respiratory cycle obtained by measurement of the respiratory state. The heartbeat interval is indicated as RRI. For the heart rate variability, CvRR, LF, HF, and LF+HF were used. The respiratory cycle is indicated as RSPI. Note that details of these physiological measures will be described later.

For the electrocardio waveform measurement, electrodes were placed under the left and right collarbones and on the left side of the body of each subject to measure the electrocardio waveform of each subject. For the respiratory state measurement, a belt was wrapped around the abdomen of each subject to measure the respiratory state.

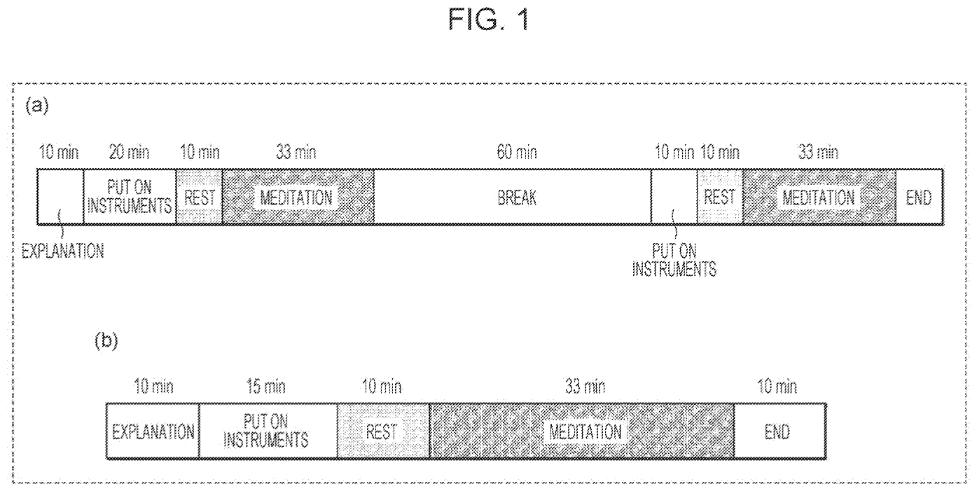

FIG. 1 is a diagram illustrating the experimental protocol. Specifically, FIG. 1(a) indicates the experimental protocol carried out with respect to the experienced subjects and FIG. 1(b) indicates the experimental protocol carried out with respect to the novice subjects.

In the experiment conducted on the experienced subjects, as illustrated in FIG. 1(a), first, the experiment is explained to the experienced subjects and the subjects are asked to put on the measuring instruments to be used in the experiment. The measuring instruments are the electrodes and belt described above or the like. Next, the subjects are asked to remain in a resting state for 10 minutes, and are then guided to meditate for 33 minutes according to voice guidance. The subjects are guided into meditation by the voice guidance, and gradually meditate. According to this protocol, the first round of the meditation experiment ends. After the first-round experiment ends, the subjects are given a 1 hour break, and then the same experiment as the first-round experiment is performed again. Note that if the subjects are guided into concentration meditation in the first-round experiment, the subjects are guided into insight meditation in the second-round experiment. Conversely, if the subjects are guided into insight meditation in the first-round experiment, the subjects are guided into concentration meditation in the second-round experiment. Note that concentration meditation and insight meditation are specific types of meditation.

In the evaluation of the meditative state of the experienced subjects, multiple types of physiological measures of the subjects obtained in each of a rest evaluation period and a meditation evaluation period are used. The rest evaluation period is the first 7 minutes and 30 seconds of the 10 minute rest period when the subjects are in a resting state.

The meditation evaluation period is the last 7 minutes and 30 seconds of the 33 minute meditation period in which the subjects are guided to meditate. The types of physiological measures are measures obtained from the electrocardio waveform and respiratory state measured by the measuring instruments described above, namely the RRI, LF, HF, LF+HF, and RSPI.

Additionally, the changes in each type of the physiological measures obtained in the rest evaluation period and the meditation evaluation period were evaluated. In other words, for each type of physiological measure, how much that physiological measure changed from the rest evaluation period to the meditation evaluation period was evaluated.

As illustrated in FIG. 1(b), the experiment conducted on the novice subjects is similar to the first-round experiment conducted on the experienced subjects above. Specifically, first, the experiment is explained to the novice subjects and the subjects are asked to put on the measuring instruments to be used in the experiment. Next, the subjects are asked to remain in a resting state for 10 minutes, and are then guided to meditate for 33 minutes according to voice guidance. In this experiment, each subject performed one of either concentration meditation or insight meditation.

In the evaluation of the meditative state of the novice subjects, multiple types of physiological measures of the subjects obtained in each of a rest evaluation period and a meditation evaluation period are used, similarly to the experienced subjects described above. Additionally, the changes in each type of the physiological measures obtained in the rest evaluation period and the meditation evaluation period were evaluated. In other words, for each type of physiological measure, how much that physiological measure changed significantly from the rest evaluation period to the meditation evaluation period was evaluated using the P-value obtained by the Wilcoxon signed-rank test.

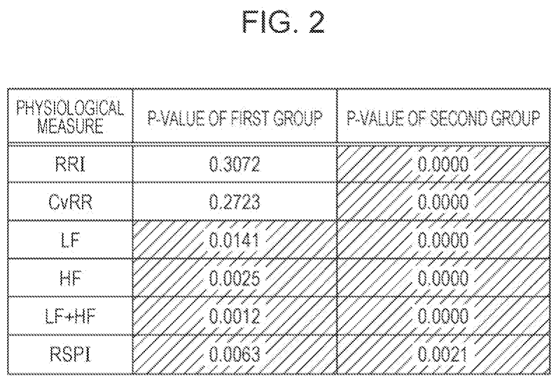

FIG. 2 is a diagram illustrating P-values of physiological measures obtained through experiment.

The P-values for each physiological measure were calculated by testing each physiological measure in each of the first group consisting of 17 subjects, each of whom is an experienced meditator, and the second group consisting of 28 persons, each of whom is a novice meditator. The P-value is the probability that there is no difference between the physiological measure in the meditation evaluation period and the physiological measure in the rest evaluation period. That is, the smaller the P-value is, the more significant a change in the physiological measure from the rest evaluation period to the meditation evaluation period is indicated. Specifically, a P-value less than or equal to 0.05 indicates a significantly large change in the physiological measure corresponding to that P-value. Note that the P-values are calculated on the basis of the results of the concentration meditation experiment and the results of the insight meditation experiment, without distinguishing between the results of each of the experiments.

As illustrated in FIG. 2, in the first group consisting of 17 persons, each of whom is an experienced meditator, LF, HF, LF+HF, and RSPI each undergo a significantly large change. On the other hand, in the second group consisting of 28 persons, each of whom is a novice meditator, RRI, CvRR, LF, HF, LF+HF, and RSPI all undergo a significantly large change.

In other words, for the novice subjects, the heart rate decreases, the heart rate variability increases, and the respiratory rate decreases in the meditation evaluation period compared to the rest evaluation period. On the other hand, for the experienced subjects, the heart rate variability excluding CvRR increases and the respiratory rate decreases in the meditation evaluation period compared to the rest evaluation period, similarly to the novice subjects. A conceivable reason for the smaller changes in the heart rate and CvRR in the experienced subjects compared to the novice subjects is that the resting state of the experienced subjects is close to a meditative state.

Therefore, the experiments described above demonstrate that physiological measures change with meditation, and specifically, a decrease in heart rate, an increase in heart rate variability, and a decrease in respiratory rate were shown to occur. Accordingly, the inventors arrived at the idea that if information indicating changes in physiological measures is fed back to a subject, the subject can be motivated to meditate.

As described above, the changes in physiological measures from the rest evaluation period to the meditation evaluation period are a decrease in heart rate, an increase in heart rate variability, and a decrease in respiratory rate. Such changes are considered to be similar to the psychological changes of relaxation or the like. That is, these changes in physiological measures are considered to be similar to the changes in physiological measures that occur when a subject is in a psychologically relaxed state. Accordingly, the inventors arrived at the idea that if information indicating psychological changes rather than physiological changes is fed back to a subject, the subject likewise can be motivated to meditate. Psychological changes are obtained from responses to a psychological questionnaire. For example, a psychological questionnaire about psychological indicators related to relaxation, such as fatigue, exhilaration, and tension/excitement, is considered useful.

Accordingly, an information processing method according to an aspect of the present disclosure is an information processing method to be performed by a computer and includes: executing acquisition processing that acquires n types (where n is an integer equal to or greater than 1) of physiological measures of a person in a state for meditation; executing determination processing that determines whether each of the acquired n types of physiological measures exceeds a threshold value corresponding to that type of physiological measure; executing derivation processing that derives a score related to a meditative state of the person on the basis of the result of the determination processing; and outputting the derived score. Note that meditation in the present disclosure may mean quieting the mind to become mindless, focusing the mind on something, closing one's eyes and letting one's thoughts run deeply and quietly, and the like. For example, meditation may include mindfulness, seated Zen meditation, yoga, and the like.

For example, if a person is simply guiding into meditation by voice guidance, the person experiencing meditation will not know how well they meditated. In particular, a beginner in meditation may give up on meditation before the effects of meditation are obtained. In the related art, no method exists to score a meditative state.

However, with the above information processing method according to an aspect of the present disclosure, a score related to the meditative state of a person is derived and outputted according to physiological measures of that person, making it possible to feed the score back to the person. As a result, it is possible to motivate a person to meditate and keep that person meditating repeatedly in an ongoing way. For example, when a person experiences meditation, the meditative state is scored and the score is fed back to the person. This arrangement increases the motivation to continue meditating. In other words, the person experiencing meditation can know how well they meditated and be motivated to meditate. As a result, the person experiencing meditation will try to continue to meditate and improve their meditation until the effects of meditation are actually felt.

One of the n types of physiological measures is the heartbeat interval of the person, and provided that α denotes the heartbeat interval of the person in a resting state and x denotes the threshold value corresponding to the heartbeat interval, the threshold value x may satisfy 1.0α≤x≤1.2α.

In the related art, a meditative state cannot be scored because there are no criteria for scoring a meditative state on the basis of a change in heartbeat interval from a resting state.

However, with the above information processing method according to an aspect of the present disclosure, the threshold value x satisfying 1.0α≤x≤1.2α is used as a scoring criterion based on heartbeat interval, and thus a meditative state can be scored appropriately on the basis of a change in heartbeat interval from a resting state.

One of the n types of physiological measures is the heart rate variability of the person, and provided that β denotes the heart rate variability of the person in a resting state and y denotes the threshold value corresponding to the heart rate variability, the threshold value y may satisfy 1.0β≤y≤2.1β.

In the related art, a meditative state cannot be scored because there are no criteria for scoring a meditative state on the basis of a change in heart rate variability from a resting state.

However, with the above information processing method according to an aspect of the present disclosure, the threshold value y satisfying 1.0β≤y≤2.1β is used as a scoring criterion based on heart rate variability, and thus a meditative state can be scored appropriately on the basis of a change in heart rate variability from a resting state.

One of the n types of physiological measures is the respiratory cycle of the person, and provided that γ denotes the respiratory cycle of the person in a resting state and z denotes the threshold value corresponding to the respiratory cycle, the threshold value z may satisfy 1.0γ≤z≤1.3γ.

In the related art, a meditative state cannot be scored because there are no criteria for scoring a meditative state on the basis of a change in respiratory cycle from a resting state.

However, with the above information processing method according to an aspect of the present disclosure, the threshold value z satisfying 1.0γ≤z≤1.3γ is used as a scoring criterion based on respiratory cycle, and thus a meditative state can be scored appropriately on the basis of a change in respiratory cycle from a resting state.

The acquisition processing, the determination processing, and the derivation processing may be executed in each of multiple periods, and in the outputting of the score, the scores derived in each of the periods may be arranged and displayed in chronological order.

In the related art, feedback is not provided on matters related to the meditative state, which changes from moment to moment while a person is experiencing meditation.

However, with the above information processing method according to an aspect of the present disclosure, scores which relate to the meditative state, and which have been derived in each of multiple periods, are arranged and displayed in chronological order. Accordingly, since a score related to the meditative state that changes from moment to moment is fed back, the person experiencing meditation can grasp their own meditative state in detail and be further motivated to meditate. The person experiencing meditation can compare their own bodily sensations to the scores arranged in chronological order.

Furthermore, in the derivation processing, an overall score may be derived according to the scores derived in each of the periods, and the information processing method may further include outputting the overall score.

With this arrangement, an overall score over a period including all of the periods is derived and outputted, thereby making it possible to score the meditative state for the whole period. As a result, the person experiencing meditation can easily get an overall grasp of how well they meditated.

In the derivation processing, the score expressed in n or (n+1) steps may be derived depending on whether each of the n types of physiological measures exceeds the threshold value corresponding to that type of physiological measure.

With this arrangement, the score is expressed in a number of steps according to the number of types (namely, n types) of physiological measures that are acquired, and thus the greater the number of physiological measures is, the more detailed is the score that can be fed back to the person experiencing meditation.

Each of the periods may partially overlap with the preceding period.

With this arrangement, even if a long time is required to acquire the physiological measures in each of the periods, scores can be expressed at short intervals because each of the periods partially overlaps with the preceding period. For example, even if each of the periods is 90 seconds, scores can be expressed at intervals of 10 seconds because each of the periods overlaps 80 seconds with the preceding period. As a result, the time resolution of the scores can be increased, and detailed change in the scores over time can be fed back to the person experiencing meditation.

The information processing method may further include: acquiring, after the person was in a state for meditation, response data indicating the result of a response by the person to a questionnaire related to psychological state; generating, on the basis of the acquired response data, psychological information indicating a change in the psychological state of the person after the person was in the state for meditation; and presenting the generated psychological information to the person.

This arrangement makes it possible to not only score the meditative state from a psychological perspective but also provide feedback to the person as psychological information from a psychological perspective, too. In other words, it is possible to provide feedback on the psychological effects of meditation. Consequently, it is possible to further motivate a person to meditate and keep that person meditating repeatedly in an ongoing way.

The questionnaire may be a questionnaire asking about the degree to which the person is relaxed, and the psychological information may indicate a change in the degree of relaxation.

With this arrangement, the psychological effects of meditation can be fed back appropriately, since the effects of meditation are easily manifested in the degree of relaxation.

The questionnaire may be a questionnaire asking about the degree of at least one psychological state of the person from among fatigue, nervousness, and exhilaration as the degree of relaxation, and the psychological information may indicate a change in the degree of psychological state for each of the at least one psychological state.

With this arrangement, since the degree of relaxation may be expressed in terms of fatigue, nervousness, and exhilaration, the psychological effects of meditation can be fed back more appropriately.

An information processing method according to an aspect of the present disclosure is an information processing method to be performed by a computer and includes: acquiring, after a person was in a state for meditation, response data indicating the result of a response by the person to a questionnaire related to psychological state; generating, on the basis of the acquired response data, psychological information indicating a change in the psychological state of the person after the person was in the state for meditation; and presenting the generated psychological information to the person.

With this arrangement, psychological information indicating a change in the psychological state of a person experiencing meditation can be fed back to that person. In other words, it is possible to provide feedback on the psychological effects of meditation. This arrangement increases the motivation to continue meditating. In other words, the person experiencing meditation can know how well they meditated and be motivated to meditate. As a result, the person experiencing meditation will try to continue to meditate and improve their meditation until the effects of meditation are actually felt. Consequently, it is possible to motivate a person to meditate and keep that person meditating repeatedly in an ongoing way.

The questionnaire may be a questionnaire asking about the degree to which the person is relaxed, and the psychological information may indicate a change in the degree of relaxation.

With this arrangement, the psychological effects of meditation can be fed back appropriately, since the effects of meditation are easily manifested in the degree of relaxation.

The questionnaire may be a questionnaire asking about the degree of at least one psychological state of the person from among fatigue, nervousness, and exhilaration as the degree of relaxation, and the psychological information may indicate a change in the degree of psychological state for each of the at least one psychological state.

An information processing method according to an aspect of the present disclosure is an information processing method to be performed by a computer and includes: executing acquisition processing that acquires n types (where n is an integer equal to or greater than 1) of physiological measures of a person in a state for meditation; executing derivation processing that derives a score related to a meditative state of the person in each of multiple periods on the basis of each of the acquired n types of physiological measures; and outputting the scores derived in each of the periods such that the scores are arranged and displayed in chronological order.

With this arrangement, a score related to the meditative state of a person is derived and outputted according to physiological measures of that person, making it possible to feed the score back to the person. As a result, it is possible to motivate a person to meditate and keep that person meditating repeatedly in an ongoing way. Scores which relate to the meditative state, and which have been derived in each of multiple periods, are arranged and displayed in chronological order. Accordingly, since a score related to the meditative state that changes from moment to moment is fed back, the person experiencing meditation can grasp their own meditative state in detail and be further motivated to meditate. The person experiencing meditation can compare their own bodily sensations to the scores arranged in chronological order.

Furthermore, in the derivation processing, an overall score may be derived according to the scores derived in each of the periods, and the information processing method may further include outputting the overall score. For example, the overall score may be the sum of the scores derived in each of the periods.

With this arrangement, an overall score over a period including all of the periods is derived and outputted, thereby making it possible to score the meditative state for the whole period. As a result, the person experiencing meditation can easily get an overall grasp of how well they meditated.

In the derivation processing, the score expressed in n or (n+1) steps may be derived.

With this arrangement, the score is expressed in a number of steps according to the number of types (namely, n types) of physiological measures that are acquired, and thus the greater the number of physiological measures is, the more detailed is the score that can be fed back to the person experiencing meditation.

With this arrangement, since the degree of relaxation may be expressed in terms of fatigue, nervousness, and exhilaration, the psychological effects of meditation can be fed back more appropriately.

It should be noted that these general or specific aspects may be implemented as a system, a method, an integrated circuit, a computer program, a computer-readable recording medium such as a CD-ROM disc, or any selective combination thereof. The recording medium may be a non-transitory recording medium.

Hereinafter, embodiments will be described specifically with reference to the drawings.

Note that the embodiments described hereinafter all illustrate general or specific examples. Features such as numerical values, shapes, materials, components, layout positions and connection states of components, steps, and the ordering of steps indicated in the following embodiments are merely examples, and are not intended to limit the present disclosure. Among the components in the following embodiments, components that are not described in the independent claim indicating the broadest concept are described as arbitrary or optional components.

Each diagram is a schematic diagram, and does not necessarily illustrate a strict representation. In each diagram, like component members are denoted with like reference signs.

### Embodiment

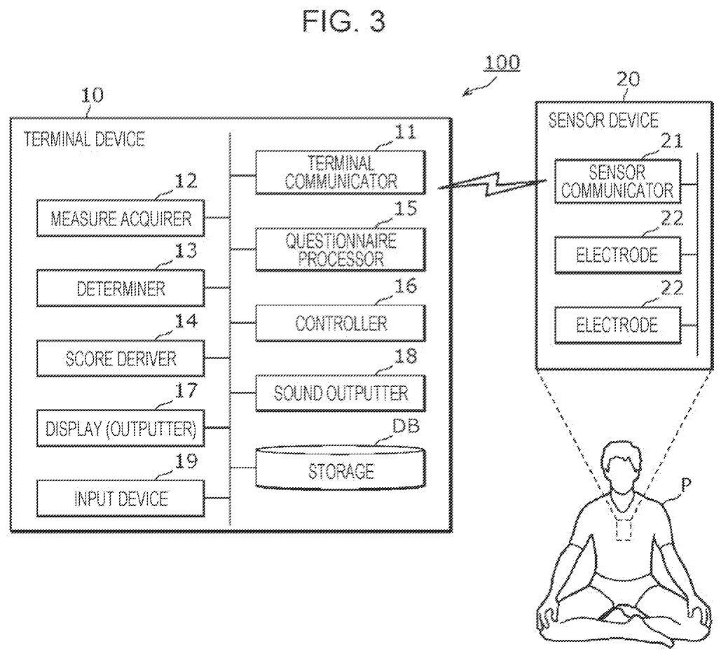

FIG. 3 is a block diagram illustrating a configuration of an information processing system according to the embodiment.

An information processing system 100 in the present embodiment is provided with a terminal device 10 and a sensor device 20.

The sensor device 20 is attached to, for example, the chest of a person P who will try to meditate, and transmits to the terminal device 10 a sensing signal, that is, an electrical signal generated by the heartbeat of the person P. This sensor device 20 is provided with a sensor communicator 21 and two electrodes 22.

The two electrodes are in contact with the chest of the person P and detect the electrical signal generated by the heartbeat of the person P. The two electrodes then output the detected electrical signal to the sensor communicator 21 as the sensing signal. Note that in the present embodiment, there are two electrodes 22, but the number of electrodes 22 is not limited to two and may also be three or more.

The sensor communicator 21 communicates wirelessly with the terminal device 10 and transmits the sensing signal described above to the terminal device 10. The wireless communication method may be Wi-Fi®, Bluetooth®, ZigBee, or specified low-power radio.

The terminal device 10 is a mobile terminal such as a smartphone or a tablet, for example. The terminal device 10 receives the sensing signal transmitted from the sensor device 20, derives a score related to the meditative state of the person P on the basis of the sensing signal, and presents the score to the person P. The terminal device 10 generates psychological information indicating a change in the psychological state of the person on the basis of a response to a questionnaire related to psychological state, and presents the psychological information to the person P. Note that the questionnaire related to psychological state described above is hereinafter also referred to as the psychological questionnaire.

This terminal device 10 is provided with a terminal communicator 11, a measure acquirer 12, a determiner 13, a score deriver 14, a questionnaire processor 15, a controller 16, a display 17, a sound outputter 18, an input device 19, and storage DB.

The storage DB is a recording medium storing, for example, guidance information indicating voice guidance and questionnaire information indicating the psychological questionnaire to be administered to the person P. The storage DB may also store a computer program. This storage DB is, for example, a hard disk drive, random access memory (RAM), read-only memory (ROM), or semiconductor memory. Note that this storage DB may be volatile or non-volatile.

The terminal communicator 11 communicates wirelessly with the sensor communicator 21 of the sensor device 20 and receives the sensing signal transmitted from the sensor communicator 21. Note that in the present embodiment, the communication between the terminal communicator 11 and the sensor communicator 21 is wireless communication, but may also be wired communication.

The measure acquirer 12 measures, on the basis of the sensing signal received by the terminal communicator 11, an electrocardio waveform (also referred to as an electrocardiogram) of the person P in a state for meditation. Note that a state for meditation includes not only the state in which the person P is meditating but also the state in which the person P is being guided into meditation, and refers to the state of the person P in the meditation evaluation period. The measure acquirer 12 calculates physiological measures from the electrocardio waveform. Physiological measures of the person P are acquired in this way. The types of physiological measures to be acquired may be multiple types rather than a single type. That is, the measure acquirer 12 executes acquisition processing that acquires n types (where n is an integer equal to or greater than 1) of physiological measures of the person P in the state for meditation. Note that in the present embodiment, the terminal device 10 is provided with the measure acquirer 12, but the sensor device 20 may also be provided with some or all of the functions of the measure acquirer 12. In one example, the sensor device 20 measures the electrocardio waveform of the person P on the basis of the sensing signal. The sensor communicator 21 of the sensor device 20 then transmits data indicating the electrocardio waveform to the terminal communicator 11.

The determiner 13 determines whether the physiological measures acquired by the measure acquirer 12 exceed threshold values. That is, the determiner 13 executes determination processing that determines whether each of the acquired n types of physiological measures exceeds a threshold value corresponding to that type of physiological measure.

The score deriver 14 derives a score on the basis of a determination result from the determiner 13. That is, the score deriver 14 executes derivation processing that derives a score related to the meditative state of the person P on the basis of the result of the determination processing described above.

The display 17 displays information such as images and text under control by the questionnaire processor 15, the controller 16, or the like. Specifically, the display 17 may be, but is not limited to, a liquid crystal display (LCD) panel or an organic light-emitting diode (OLED) display panel, for example. Note that in the present embodiment, the display 17 is an example of an outputter that outputs a score related to meditative state and an example of an outputter that presents psychological information described later to the person P.

The sound outputter 18 is a speaker, for example, that outputs sound, music, or speech under control by the controller 16.

The input device 19 has buttons and the like for accepting input operations by the person P, and outputs an input signal corresponding to an input operation to the questionnaire processor 15, the controller 16, or the like. Note that the display 17 and the input device 19 may also be integrated to form a touch panel.

The questionnaire processor 15 displays the psychological questionnaire on the display 17 after the person P was in the state for meditation, or in other words after the meditation period. In addition, the questionnaire processor 15 acquires response data indicating the result of a response to the psychological questionnaire, namely an input signal corresponding to an input operation performed on the input device 19 by the person P. Furthermore, the questionnaire processor 15 generates, on the basis of the acquired response data, psychological information indicating a change in the psychological state of the person P after the person P was in the state for meditation. The questionnaire processor 15 then displays the generated psychological information on the display 17 to thereby present the psychological information to the person P.

The controller 16 controls each component of the terminal device 10 other than the controller 16 itself. For example, the controller 16 reads out guidance information from the storage DB and causes the sound outputter 18 to output voice guidance indicated by the guidance information. The controller 16 may be configured as a central processing unit (CPU) or a processor. In this case, for example, the controller 16 may read out a meditation application program, which is a computer program stored in the storage DB, and execute the meditation application program to control the above components.

Note that the meditation application program is a program causing a computer to execute acquisition processing that acquires n types of physiological measures of the person P in a state for meditation; execute determination processing that determines whether each of the acquired n types of physiological measures exceeds a threshold value corresponding to that type of physiological measure; execute derivation processing that derives a score related to the meditative state of the person P on the basis of the result of the determination processing; and output the derived score. The meditation application program is a program causing a computer to acquire, after the person P was in the state for meditation, response data indicating the result of a response by the person P to a questionnaire related to psychological state; generate, on the basis of the acquired response data, psychological information indicating a change in the psychological state of the person P after the person P was in the state for meditation; and present the generated psychological information to the person P. Note that this meditation application program is hereinafter also referred to as the meditation app.

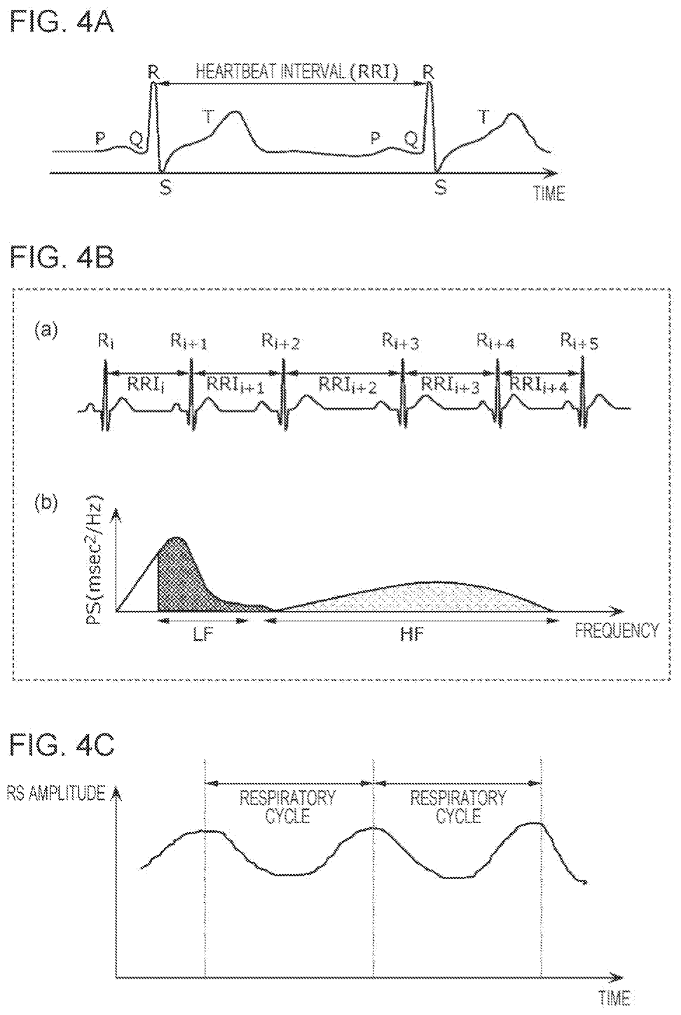

FIG. 4A is a diagram illustrating an electrocardio waveform for explaining heartbeat interval. Note that in FIG. 4A, the horizontal axis represents time and the vertical axis represents electric potential difference.

The electrocardio waveform indicated by the sensing signal is also referred to as an electrocardiogram. The electrocardio waveform includes P waves that reflect electrical excitation of the atria, Q, R, and S waves that reflect electrical excitation of the ventricles, and T waves that reflect the process of repolarization of the excited ventricular myocardial cells. Among these waves, the R waves have the tallest wave height (that is, electric potential difference) and are the most robust against noise such as myoelectric potential.

The measure acquirer 12 measures this electrocardio waveform and detects two consecutive R waves in the electrocardio waveform. Note that a known technique such as the Pan & Tompkins method, for example, may be used to detect R waves. Additionally, the measure acquirer 12 specifies the interval between the peaks of the two R waves as the heartbeat interval (the R-R interval, or RRI). With this arrangement, the RRI is acquired by the measure acquirer 12 as one type of physiological measure. Note that heart rate is the number of beats per minute, for example, which is calculated by dividing 60 seconds by the number of seconds in the RRI.

FIG. 4B is a diagram for explaining heart rate variability. Note that FIG. 4B(a) is a graph illustrating an electrocardio waveform, and in this graph, the horizontal axis represents time and the vertical axis represents electric potential difference. Also, FIG. 4B(b) is a graph illustrating the spectrum of heart rate variability, and in this graph, the horizontal axis represents frequency and vertical axis represents power.

As illustrated in FIG. 4B(a), the electrocardio waveform includes Ri, Rᵢ₊₁, Rᵢ₊₂, . . . , and Rᵢ₊₅ as consecutive R waves. The intervals of these mutually adjacent R waves are RRIᵢ, RRIᵢ₊₁, RRIᵢ₊₂, . . . , and RRIᵢ₊₄. These intervals are all the RRI, but are not constant. Therefore, the RRI fluctuates. Note that fluctuations in the RRI are also referred to as heartbeat fluctuations or heart rate variability.

The measure acquirer 12 acquires each of the coefficient of variation of R-R intervals (CvRR), low frequency (LF), high frequency (HF), and LF+HF as the heart rate variability. With this arrangement, CvRR, LF, HF, and LF+HF are each acquired as one type of physiological measure.

CvRR is the coefficient of variation of the heart rate variability. The measure acquirer 12 calculates CvRR according to the formula “CvRR=(standard deviation SD of RRI over any given time period)/(average of RRI over any given time period)”, for example. That is, the measure acquirer 12 calculates CvRR by normalizing the standard deviation SD of the RRI over any given time period by the mean of the RRI over any given time period.

In the acquisition of LF, HF, and LF+HF, as illustrated in FIG. 4B(b), the measure acquirer 12 performs frequency analysis on the RRI time series data using the fast Fourier transform (FFT). LF and HF are calculated from the power spectrum obtained by the frequency analysis. LF is the integral value of the power spectrum in the low frequency range from 0.04 Hz to 0.14 Hz, and is thought to reflect the amount of sympathetic and parasympathetic nervous activity. HF is the integral value of the power spectrum in the high frequency range from 0.14 Hz to 0.4 Hz, and is thought to reflect the amount of parasympathetic nervous activity. Note that the frequency transform of the FFT may be performed at 5-second intervals. LF+HF is calculated by adding HF to LF.

FIG. 4C is a diagram for explaining respiratory cycle. Note that FIG. 4C is a graph illustrating change over time in the RS amplitude, which is the amplitude between the R and S waves in the electrocardio waveform, and in this graph, the horizontal axis represents time and the vertical axis represents RS amplitude.

The measure acquirer 12 extracts the respiratory cycle from the electrocardio waveform. Note that the respiratory cycle is also referred to as the respiration signal peak interval (RSPI). A method for extracting the respiratory cycle from the electrocardio waveform is described in the following document: Charlton P H et al. Extraction of respiratory signals from the electrocardiogram and photoplethysmogram: technical and physiological determinants. Physiological Measurement. 2017 May;38(5):669-690. doi: https://doi.org/10.1088/1361-6579/aa670e.

The measure acquirer 12 measures the RS amplitudes indicated by the electrocardio waveform for each beat and analyzes the time variability of the RS amplitudes. As illustrated in FIG. 4C, the RS amplitudes vary according to chest expansion due to respiration. In other words, the RS amplitudes are modulated according to the state of respiration. The measure acquirer 12 extracts the respiratory cycle by detecting peaks from the waveform of the RS amplitudes illustrated in FIG. 4C and specifying the interval between two consecutive peaks.

As above, the measure acquirer 12 acquires six types of physiological measures, namely the RRI, CvRR, LF, HF, LF+HF, and RSPI, but not all six types of physiological measures have to be acquired, and one or more types of physiological measures may simply be acquired.

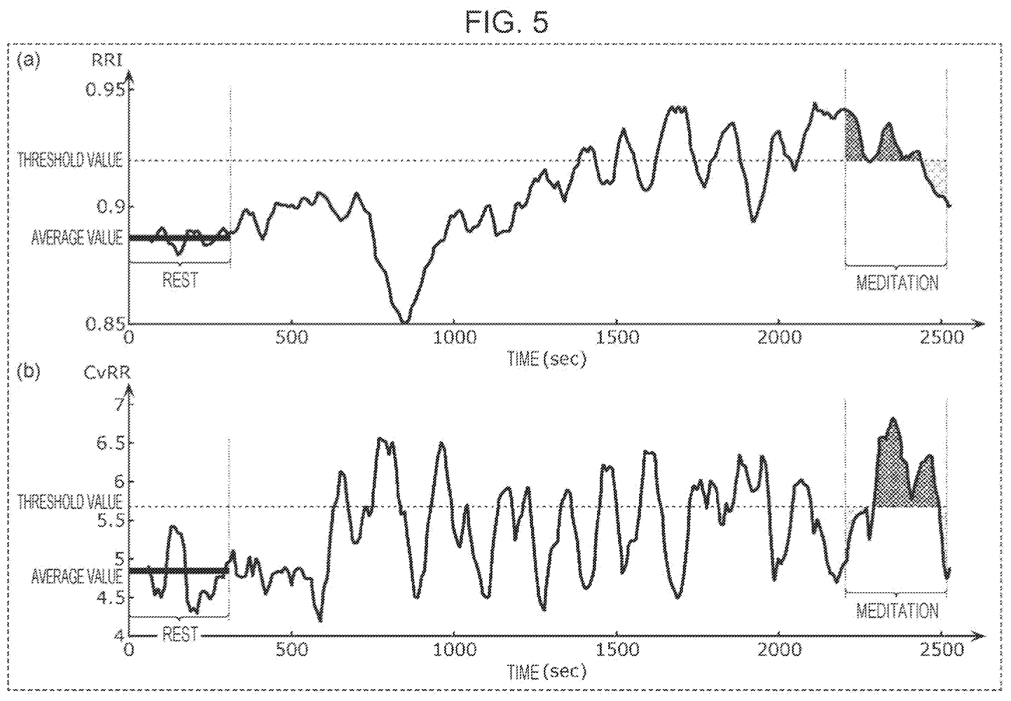

FIG. 5 is a diagram for explaining the derivation of the score. Note that FIG. 5(a) is a graph illustrating the change over time in the RRI, and in this graph, the horizontal axis represents time and the vertical axis represents RRI. Also, FIG. 5(b) is a graph illustrating the change over time in the CvRR, and in this graph, the horizontal axis represents time and the vertical axis represents CvRR.

The controller 16 prompts the person P to begin the resting state by controlling the display 17 or the sound outputter 18, for example, and puts the person P into the resting state in the rest period. For example, the controller 16 displays a message on the display 17 prompting the user to begin the resting state, or causes the sound outputter 18 to output voice guidance prompting the same. Thereafter, in the meditation period, the controller 16 guides the person P into meditation by causing the sound outputter 18 to output voice guidance. The rest period is 10 minutes like the experiments above, for example, and the meditation period is 33 minutes like the experiments above, for example. In the rest period and the meditation period, the controller 16 causes the terminal communicator 11 to receive the sensing signal from the sensor device 20 and causes the measure acquirer 12 to execute acquisition of various physiological measures based on the sensing signal.

For example, as illustrated in FIG. 5(a), the measure acquirer 12 acquires the RRI in each of the rest period and the meditation period described above. The determiner 13 calculates the mean of the RRI in the rest evaluation period, which is the first 5 minutes of the rest period, for example. The determiner 13 then calculates a threshold value for the RRI by multiplying the mean of the RRI by a threshold parameter corresponding to the RRI. Note that the threshold parameter is a value predetermined with respect to the RRI.

Next, the determiner 13 determines whether the RRI exceeds the threshold value described above in the meditation evaluation period, which is the last 5 minutes of the meditation period, for example. The score deriver 14 identifies a period in which the RRI is determined to exceed the threshold value as a high RRI period, and calculates the ratio of the high RRI period to the meditation evaluation period as the score. The ratio may be calculated as a percentage. In this case, the score based on the RRI is a numerical value from 1 to 100.

Similarly, as illustrated in FIG. 5(b), the measure acquirer 12 acquires the CvRR in each of the rest period and the meditation period described above. The determiner 13 calculates the mean of the CvRR in the rest evaluation period, which is the first 5 minutes of the rest period, for example. The determiner 13 then calculates a threshold value for the CvRR by multiplying the mean of the CvRR by a threshold parameter corresponding to the CvRR. Note that the threshold parameter is a value predetermined with respect to the CvRR.

Next, the determiner 13 determines whether the CvRR exceeds the threshold value described above in the meditation evaluation period, which is the last 5 minutes of the meditation period, for example. The score deriver 14 identifies a period in which the CvRR is determined to exceed the threshold value as a high CvRR period, and calculates the ratio of the high CvRR period to the meditation evaluation period as the score. The ratio may be calculated as a percentage, for example. In this case, the score based on the CvRR is a numerical value from 1 to 100.

For each of the physiological measures of the LF, HF, LF+HF, and RSPI, a score based on the physiological measure is calculated in a manner similar to the RRI and CvRR above. Note that these scores calculated for each type of physiological measure are hereinafter also referred to as individual scores. In the example illustrated in FIG. 5, physiological measures are acquired in the entire period of each of the rest period and the meditation period, but the measure acquirer 12 may also acquire physiological measures in the rest evaluation period and the meditation evaluation period.

The score deriver 14 may also calculate a single meditation score from the individual scores calculated in this way. For example, the score deriver 14 calculates the meditation score by weighted addition of the individual scores. In the weighted addition, the score deriver 14 multiplies each of the calculated individual scores by a coefficient and sums the resulting products. The coefficients are each less than 1, and together add up to 1. If the coefficients are equal to each other, the meditation score derived by the weighted addition will be the average of the individual scores. The score deriver 14 may also calculate the meditation score by simply adding up the individual scores.

The controller 16 may display the calculated individual scores on the display 17 or display the meditation score on the display 17. That is, the individual scores or the meditation score is outputted.

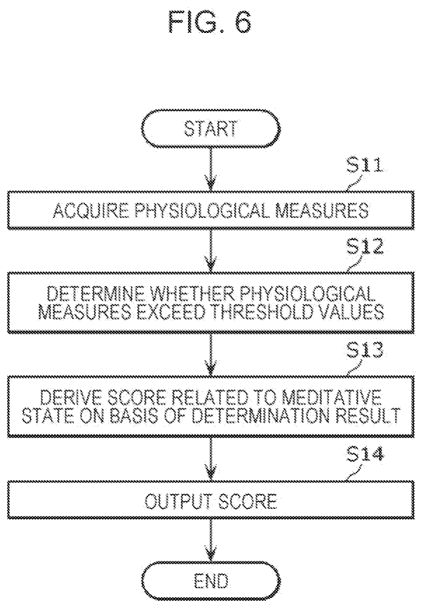

FIG. 6 is a flowchart illustrating an example of general score-related processing operations by the terminal device 10.

In the present embodiment, the terminal device 10 executes acquisition processing that acquires n types of physiological measures of the person P in the state for meditation (step S11). Thereafter, the terminal device 10 executes determination processing that determines whether each of the acquired n types of physiological measures exceeds the threshold value corresponding to that type of physiological measure (step S12). Thereafter, the terminal device 10 executes derivation processing that derives a score related to the meditative state of the person P on the basis of the result of the determination processing (step S13), and outputs the derived score (step S14). Note that the score may be the individual scores or the meditation score. The score may also be a sub-score to be described later.

With this arrangement, a score related to the meditative state of the person P is derived and outputted according to physiological measures of the person P, making it possible to feed the score back to the person P. As a result, it is possible to motivate the person P to meditate and keep the person P meditating repeatedly in an ongoing way. For example, when the person P experiences meditation, the meditative state is scored and the score is fed back to the person. This arrangement increases the motivation to continue meditating. In other words, the person P experiencing meditation can know how well they meditated and be motivated to meditate. As a result, the person P experiencing meditation will try to continue to meditate and improve their meditation until the effects of meditation are actually felt.

Note that the method of scoring the meditative state in the present embodiment is a method based on the results of the experiment with novice subjects, and is a method of scoring that particularly suited to persons who need to meditate in an ongoing way. By scoring and providing feedback to the meditator after a meditation experience, the meditator can obtain a sense of accomplishment and be motivated to continue meditating. As described above, the results of the experiment show that during meditation, each of the heart rate variability and the respiratory cycle other than CvRR tend to increase, even in experienced meditators. Therefore, the method of scoring the meditative state in the present disclosure is also applicable to experienced meditators.

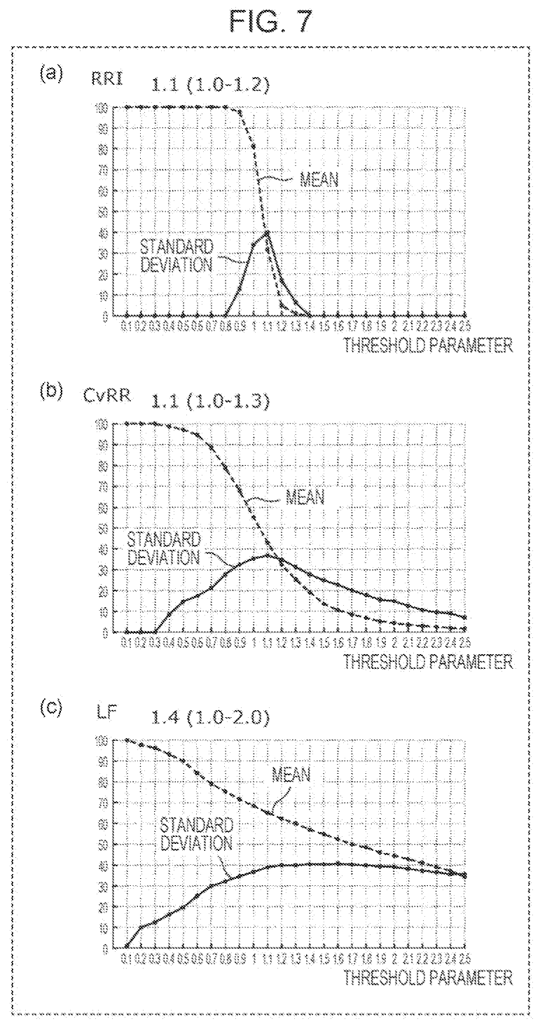

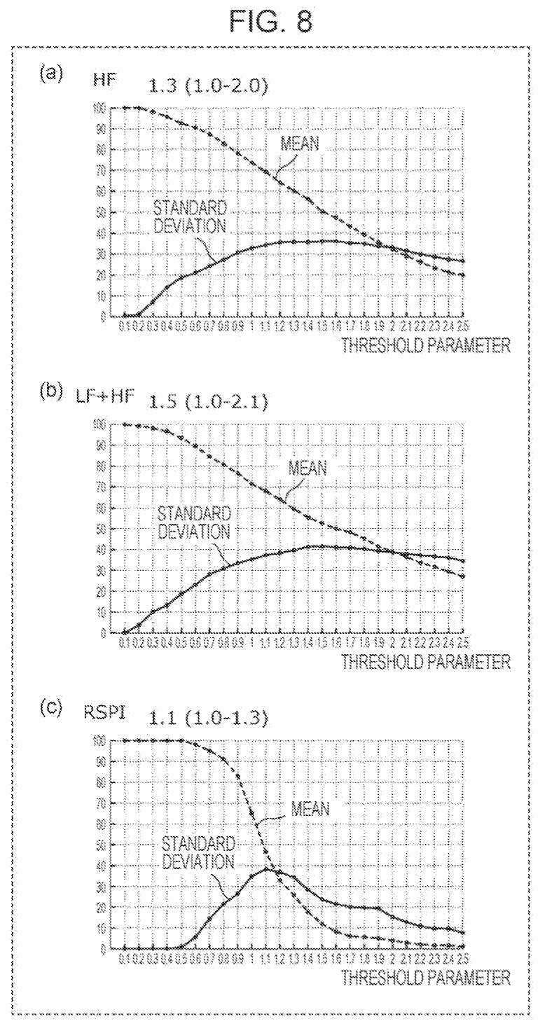

When scores are derived for multiple persons, the scores for the persons should be spread out appropriately. Accordingly, based on the results of the experiment with novice subjects described above, appropriate threshold parameters are determined in advance so that the scores for multiple persons can be spread out appropriately. FIGS. 7 and 8 will be referenced to describe the determination of appropriate threshold parameters.

FIG. 7 is a diagram for explaining threshold parameters of physiological measures. Specifically, FIG. 7(a) is a graph illustrating changes depending on the threshold parameter for each of the mean and the standard deviation of the score based on the RRI, and in this graph, the horizontal axis represents the threshold parameter and the vertical axis represents the mean and the standard deviation. Also, FIG. 7(b) is a graph illustrating changes depending on the threshold parameter for each of the mean and the standard deviation of the score based on the CvRR, and in this graph, the horizontal axis represents the threshold parameter and the vertical axis represents the mean and the standard deviation. Also, FIG. 7(c) is a graph illustrating changes depending on the threshold parameter for each of the mean and the standard deviation of the score based on the LF, and in this graph, the horizontal axis represents the threshold parameter and the vertical axis represents the mean and the standard deviation.

For the RRI, as illustrated in FIG. 7(a), if the threshold parameter is too large, the mean of the scores derived for multiple subjects goes to 0, and conversely, if the threshold parameter is too small, the mean of the scores derived for multiple subjects goes to 100. When the threshold parameter is 1.1, the standard deviation is maximized and the scores for the subjects are widely dispersed. Therefore, 1.1, or a numerical value in the range equal to or greater than 1.0 and less than or equal to 1.2, is used as an appropriate threshold parameter for the RRI.

In other words, in the present embodiment, one of the n types of physiological measures is the heartbeat interval of the person P, and provided that α denotes the heartbeat interval of the person P in a resting state and x denotes the threshold value corresponding to the heartbeat interval, the threshold value x satisfies 1.0α≤x≤1.2α. With this arrangement, the meditative state of the person P can be scored appropriately on the basis of a change in the heartbeat interval from the resting state.

Similarly, for the CvRR, as illustrated in FIG. 7(b), if the threshold parameter is too large, the mean of the scores derived for multiple subjects approaches 0, and conversely, if the threshold parameter is too small, the mean of the scores derived for multiple subjects goes to 100. When the threshold parameter is 1.1, the standard deviation is maximized and the scores for the subjects are widely dispersed. Therefore, 1.1, or a numerical value in the range equal to or greater than 1.0 and less than or equal to 1.3, is used as an appropriate threshold parameter for the CvRR.

Similarly, for the LF, as illustrated in FIG. 7(c), if the threshold parameter is too large, the mean of the scores derived for multiple subjects approaches 0, and conversely, if the threshold parameter is too small, the mean of the scores derived for multiple subjects goes to 100. When the threshold parameter is 1.4, the standard deviation is maximized and the scores for the subjects are widely dispersed. Therefore, 1.4, or a numerical value in the range equal to or greater than 1.0 and less than or equal to 2.0, is used as an appropriate threshold parameter for the LF.

The range of the threshold parameter for each of the RRI, CvRR, and LF may be set according to the shape of the distribution of the standard deviation corresponding to that threshold parameter. For example, the range of the threshold parameter for each physiological measure may be set so that the shorter the half-width of the standard deviation corresponding to that physiological measure is, the narrower the range is, and the longer the half-width is, the wider the range is. In this case, the median of the threshold parameter range may be the numerical value corresponding to the maximum standard deviation. The threshold parameter range may be set to a range corresponding to an average score from 30 to 70, for example.

FIG. 8 is a diagram for explaining threshold parameters of the other physiological measures. Specifically, FIG. 8(a) is a graph illustrating changes depending on the threshold parameter for each of the mean and the standard deviation of the score based on the HF, and in this graph, the horizontal axis represents the threshold parameter and the vertical axis represents the mean and the standard deviation. Also, FIG. 8(b) is a graph illustrating changes depending on the threshold parameter for each of the mean and the standard deviation of the score based on the LF+HF, and in this graph, the horizontal axis represents the threshold parameter and the vertical axis represents the mean and the standard deviation. Also, FIG. 8(c) is a graph illustrating changes depending on the threshold parameter for each of the mean and the standard deviation of the score based on the RSPI, and in this graph, the horizontal axis represents the threshold parameter and the vertical axis represents the mean and the standard deviation.

When scores are derived for multiple persons, the scores for the persons should be spread out appropriately with respect to the threshold parameters for each of HF, LF+HF, and RSPI, in a manner similar to the RRI, CvRR, and LF. Accordingly, these threshold parameters likewise are determined on the basis of the results of the experiment with novice subjects described above.

For the HF, as illustrated in FIG. 8(a), if the threshold parameter is too large, the mean of the scores derived for multiple subjects approaches 0, and conversely, if the threshold parameter is too small, the mean of the scores derived for multiple subjects goes to 100. When the threshold parameter is 1.3, the standard deviation is maximized and the scores for the subjects are widely dispersed. Therefore, 1.3, or a numerical value in the range equal to or greater than 1.0 and less than or equal to 2.0, is used as an appropriate threshold parameter for the HF.

Similarly, for the LF+HF, as illustrated in FIG. 8(b), if the threshold parameter is too large, the mean of the scores derived for multiple subjects approaches 0, and conversely, if the threshold parameter is too small, the mean of the scores derived for multiple subjects goes to 100. When the threshold parameter is 1.5, the standard deviation is maximized and the scores for the subjects are widely dispersed. Therefore, 1.5, or a numerical value in the range equal to or greater than 1.0 and less than or equal to 2.1, is used as an appropriate threshold parameter for the LF+HF.

As in FIGS. 7(b) and 7(c), and FIGS. 8(a) and 8(b), in the present embodiment, one of the n types of physiological measures is the heart rate variability of the person P, and provided that β denotes the heart rate variability of the person P in the resting state and y denotes the threshold value corresponding to the heart rate variability, the threshold value y satisfies 1.0β≤y≤2.1β. With this arrangement, the meditative state of the person P can be scored appropriately on the basis of a change in the heart rate variability from the resting state.

For the RSPI, as illustrated in FIG. 8(c), if the threshold parameter is too large, the mean of the scores derived for multiple subjects approaches 0, and conversely, if the threshold parameter is too small, the mean of the scores derived for multiple subjects goes to 100. When the threshold parameter is 1.1, the standard deviation is maximized and the scores for the subjects are widely dispersed. Therefore, 1.1, or a numerical value in the range equal to or greater than 1.0 and less than or equal to 1.3, is used as an appropriate threshold parameter for the RSPI.

That is, in the present embodiment, one of the n types of physiological measures is the respiratory cycle of the person P, and provided that γ denotes the respiratory cycle of the person P in the resting state and z denotes the threshold value corresponding to the respiratory cycle, the threshold value z satisfies 1.0γ≤z≤1.3γ. With this arrangement, the meditative state of the person P can be scored appropriately on the basis of a change in the respiratory cycle from the resting state.

The range of the threshold parameter for each of the HF, LF+HF, and RSPI likewise may be set according to the shape of the distribution of the standard deviation corresponding to the threshold parameter, in a manner similar to the RRI, CvRR, and LF. For example, the range of the threshold parameter for each physiological measure may be set so that the shorter the half-width of the standard deviation corresponding to that physiological measure is, the narrower the range is, and the longer the half-width is, the wider the range is. In this case, the median of the threshold parameter range may be the numerical value corresponding to the maximum standard deviation. The threshold parameter range may be set to a range corresponding to an average score from 30 to 70, for example.

In this way, a threshold parameter is determined for each of the physiological measures of the RRI, CvRR, LF, HF, LF+HF, and RSPI. As a result, the terminal device 10 derives a score for each of the multiple types of physiological measures described above, as in the example illustrated in FIG. 5.

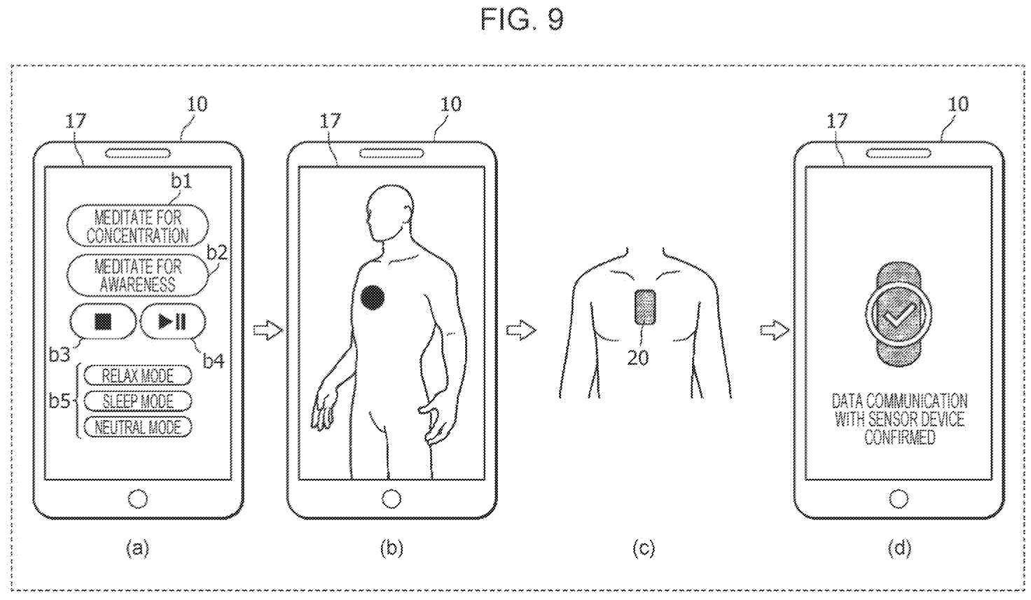

FIG. 9 is a diagram illustrating an example of screen transitions on the terminal device 10.

The controller 16 of the terminal device 10 reads out the meditation app stored in the storage DB, for example, and initiates execution of the meditation app. First, as illustrated FIG. 9(a), the controller 16 displays an operating screen on the display 17. The operating screen includes a button b1 labeled “Meditate for Concentration”, a button b2 labeled “Meditate for Awareness”, a button b3 for stopping guidance into meditation, and a button b4 for starting, resuming, or pausing guidance into meditation. The operating screen may also include buttons b5 for selecting modes such as “Relax Mode”, “Sleep Mode”, and “Neutral Mode”. Note that these modes are modes of the environment in the meditation space, which is the space around the person P. That is, if one of the modes is selected, environmental properties such as the lighting, acoustics, and fragrance of the meditation space are controlled according to the selected mode.

When starting meditation, the person P selects the button b1 or b2 on the operating screen. Thereafter, the person P selects the button b4.

If the button b1 is selected, the input device 19 accepts the selection operation on the button b1 by the person P and outputs a signal corresponding to the selection operation to the controller 16. The controller 16, upon obtaining the signal, reads out guidance information associated with “Meditate for Concentration” from the storage DB and causes the sound outputter 18 to output voice guidance indicated by the guidance information, for example. The controller 16 may also execute environmental control according to an environmental mode associated with “Meditate for Concentration”. Note that meditation for concentration is a type of meditation in which the person P concentrates awareness on one of the five senses, for example.

If the button b2 is selected, the input device 19 accepts the selection operation on the button b2 by the person P and outputs a signal corresponding to the selection operation to the controller 16. The controller 16, upon obtaining the signal, reads out guidance information associated with “Meditate for Awareness” from the storage DB and causes the sound outputter 18 to output voice guidance indicated by the guidance information, for example. The controller 16 may also execute environmental control according to an environmental mode associated with “Meditate for Awareness”. Note that meditation for awareness is a type of meditation in which the person P concentrates awareness evenly on each of the five senses, for example.

Next, the controller 16 of the terminal device 10 displays, according to the meditation app, a wearing procedure screen on the display 17 as illustrated in FIG. 9(b). The wearing procedure screen shows how to put on the sensor device 20. For example, the wearing procedure screen illustrates where the sensor device 20 is to be applied on the body of the person P. At this time, the controller 16 may also cause the sound outputter 18 to output voice guidance such as “Please apply the sensor device to your chest”. The person P follows the instructions shown on the wearing procedure screen and applies the sensor device 20 to their chest, as illustrated in FIG. 9(c).

Next, the controller 16 confirms communication between the terminal communicator 11 and the sensor communicator 21 of the sensor device 20. If communication is determined to be possible, the controller 16 displays a normal communication confirmation screen on the display 17, as illustrated in FIG. 9(d). The normal communication confirmation screen informs the person P that communication is possible between the sensor device 20 and the terminal device 10.

Next, the controller 16 instructs the person P to straighten their posture and be at rest by causing the sound outputter 18 to output voice guidance, for example. At this time, the rest period described above begins.

Thereafter, in the rest evaluation period that is a part of the rest period, the controller 16 causes the terminal communicator 11 to receive a sensing signal from the sensor device 20 and causes the measure acquirer 12 to measure an electrocardio waveform based on the sensing signal. The rest evaluation period is 5 minutes in the example described above, but may be from 1 to 10 minutes, and may also be from 3 to 5 minutes. Note that the controller 16 may also cause the measure acquirer 12 to start measurement of the electrocardio waveform immediately after communication between the terminal communicator 11 and the sensor communicator 21 is confirmed. The controller 16 may also cause the sound outputter 18 to output a message as speech informing the person P that an electrocardio waveform is to be measured in the rest evaluation period.

Next, after the rest period ends, the controller 16 guides the person P into meditation by causing the sound outputter 18 to output voice guidance for meditation. At this time, the meditation period described above begins. Thereafter, in the meditation evaluation period that is a part of the meditation period, the controller 16 causes the terminal communicator 11 to receive a sensing signal from the sensor device 20 and causes the measure acquirer 12 to measure an electrocardio waveform based on the sensing signal. The meditation evaluation period is 5 minutes in the example described above, but may be from 1 to 10 minutes, and may also be from 3 to 5 minutes.

The measure acquirer 12 acquires multiple types of physiological measures on the basis of the electrocardio waveform measured in each of the rest evaluation period and the meditation evaluation period. The determiner 13 and the score deriver 14 derive a score related to the meditative state of the person P according to the score derivation method illustrated in FIG. 5. That is, the determiner 13 calculates the mean of each physiological measure in the rest evaluation period and multiplies the mean of that physiological measure by a threshold parameter to calculate the threshold value for that physiological measure. Thereafter, the determiner 13 determines whether that physiological measure exceeds the above threshold value in the meditation evaluation period. The score deriver 14 identifies a period in which that physiological measure is determined to exceed the threshold value as a high physiological measure period, and calculates the ratio of the high physiological measure period to the meditation evaluation period as the score.

At this point, after the meditation period described above ends, the questionnaire processor 15 of the terminal device 10 reads out questionnaire information from the storage DB and displays on the display 17 a psychological questionnaire indicated by the questionnaire information. The psychological questionnaire is a questionnaire about feelings of fatigue, exhilaration, tension/excitement, and the like. Fatigue, exhilaration, and tension/excitement are each a psychological state related to relaxation, and these psychological states are also referred to as psychological indicators. Tension/excitement is also referred to as nervousness.

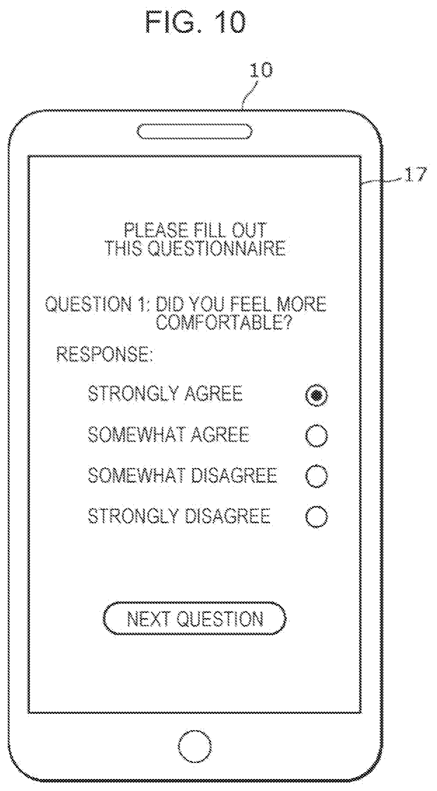

FIG. 10 is a diagram illustrating an example of a questionnaire screen.

The questionnaire processor 15 displays the questionnaire screen illustrated in FIG. 10, for example, on the display 17 on the basis of the questionnaire information. For example, the questionnaire screen includes a question and choices for responding to the question. The question is a question related to exhilaration, for example, and specifically is a question such as “Did you feel more comfortable?” The choices for responding to the question may be “Strongly agree”, “Somewhat agree”, “Somewhat disagree”, and “Strongly disagree”, or the like. The person P selects one of the choices as the response to the question. The input device 19 accepts the response selection operation by the person P and outputs response data indicating the selected response to the questionnaire processor 15.

The questionnaire processor 15 acquires the response data and specifies a number of points for the response indicated by the response data. For example, 4 points are preset with respect to the choice “Strongly agree”. Similarly, 3 points are preset with respect to the choice “Somewhat agree”, 2 points are preset with respect to the choice “Somewhat disagree”, and 1 point is preset with respect to the choice “Strongly disagree”. Consequently, if the response indicated by the response data from the input device 19 is the choice “Strongly agree”, the questionnaire processor 15 specifies 4 points preset with respect to that choice.

The question related to exhilaration may be not only the question in the above example, but also a question such as “Do you feel invigorated?” That is, the questionnaire processor 15 displays questions regarding exhilaration, such as “Did you feel more comfortable?” and “Do you feel invigorated?”, on the display 17, and acquires response data for the questions. Thereafter, the questionnaire processor 15 calculates a total number of points for exhilaration by specifying and totaling the points set with respect to the responses indicated by the response data.

The questionnaire processor 15 displays questions related to fatigue on the display 17 in a manner similar to exhilaration. The questions related to fatigue may be, for example, “Is it too much trouble?” and “Do you not want to do anything?” The choices for responding to these questions may be “Strongly agree”, “Somewhat agree”, “Somewhat disagree”, and “Strongly disagree”, similarly to exhilaration. The questionnaire processor 15 acquires response data to these questions and calculates a total number of points for fatigue by specifying and totaling the points set with respect to the responses indicated by the response data.

Similarly, the questionnaire processor 15 displays questions related to nervousness on the display 17. The questions related to nervousness may be, for example, “Do you feel anxious?” and “Are you excited?” The choices for responding to these questions may be “Strongly agree”, “Somewhat agree”, “Somewhat disagree”, and “Strongly disagree”, similarly to exhilaration. The questionnaire processor 15 acquires response data to these questions and calculates a total number of points for nervousness by specifying and totaling the points set with respect to the responses indicated by the response data.

The total number of points for fatigue, the total number of points for exhilaration, and the total number of points for nervousness calculated as above are psychological effects of meditation, and hereinafter also referred to as the psychological score or psychological information. The controller 16 displays the meditation score derived by the score deriver 14 and the psychological score calculated by the questionnaire processor 15 on the display 17.

In this way, in the present embodiment, the questionnaire processor 15 acquires response data indicating the result of a response by the person P to the psychological questionnaire after the person P was in the state for meditation. The questionnaire processor 15 then generates, on the basis of the acquired response data, psychological information indicating a change in the psychological state of the person P after the person P was in the state for meditation. The display 17 displays the generated psychological information to thereby present the psychological information to the person P. This arrangement makes it possible to not only score the meditative state from a psychological perspective but also provide feedback to the person P as psychological information from a psychological perspective, too. In other words, it is possible to provide feedback on the psychological effects of meditation. Consequently, it is possible to further motivate the person P to meditate and keep the person P meditating repeatedly in an ongoing way.

The psychological questionnaire is a questionnaire asking about the degree to which the person P is relaxed, and the psychological information indicates a change in the degree of relaxation. With this arrangement, the psychological effects of meditation can be fed back appropriately, since the effects of meditation are easily manifested in the degree of relaxation.

The psychological questionnaire is a questionnaire asking about the degree of at least one psychological state of the person P from among fatigue, nervousness, and exhilaration as the degree of relaxation, and the psychological information indicates a change in the degree of psychological state for each of the at least one psychological state. With this arrangement, since the degree of relaxation may be expressed in terms of fatigue, nervousness, and exhilaration, the psychological effects of meditation can be fed back more appropriately.

In the present embodiment, scoring the meditative state based on changes in physiological measures and scoring the meditative state based on psychological change are thought to make it possible to provide more appropriate feedback regarding the effects of meditation on the meditator. This arrangement makes it possible for the meditator to actually feel the effects of meditation more easily and increase the motivation to continue meditating.

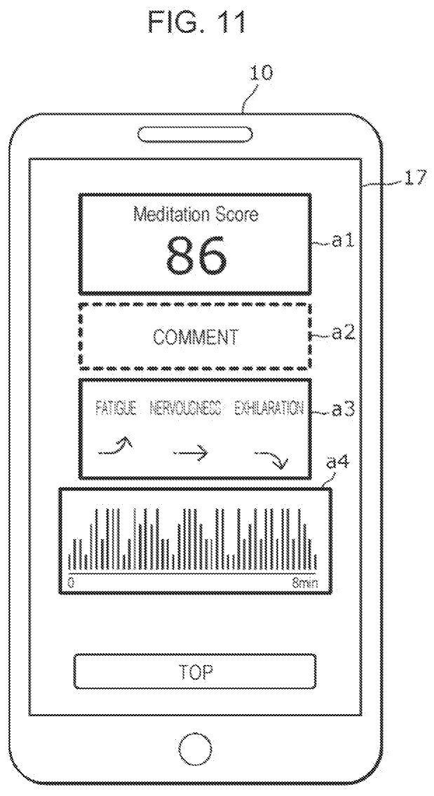

FIG. 11 is a diagram illustrating an example of a results display screen.

As illustrated in FIG. 11, the controller 16 displays, on the display 17, a results display screen indicating the meditation score and the psychological score described above. That is, the terminal device 10 feeds back the results of meditation to the person P.

The results display screen includes a meditation score area a1, a comment area a2, a psychological score area a3, and a meditation change graph area a4.

In the meditation score area a1, the meditation score is displayed as a numerical value from 0 to 100. As described above, the score deriver 14 derives an individual score for each of the multiple types of physiological measures, and then calculates the meditation score by weighted addition of the individual scores. The controller 16 displays the derived meditation score in the meditation score area a1.

In the psychological score area a3, the psychological score for fatigue, the psychological score for nervousness, and the psychological score for exhilaration are each displayed as an arrow. The higher the psychological score is, the more the arrow points upward, and conversely, the lower the psychological score is, the more the arrow points downward. For example, the controller 16 determines the form of the arrow for each of the three psychological scores according to the magnitude of that psychological score, and displays the arrow in the determined form in the psychological score area a3. Note that the psychological score is also referred to as the psychological information as described above. The psychological score may also be said to indicate the degree of improvement through meditation in the psychological state of the person P.

In the comment area a2, a comment corresponding to the meditation score and each psychological score is displayed. For example, various comment information is stored in the storage DB. Each piece of comment information is associated with the meditation score and each psychological score. The controller 16 acquires, from the storage DB, comment information associated with the meditation score calculated by the score deriver 14 and each psychological score calculated by the questionnaire processor 15. The controller 16 then displays the comment indicated by the comment information in the comment area a2. The displayed comment is a comment related to the effects of meditation or areas of improvement, for example.

In the meditation change graph area a4, a graph indicating change in the meditative state is displayed. Note that in this graph, the horizontal axis represents time and the vertical axis represents a sub-score. Forming, the meditation evaluation period includes evaluation sub-periods. For each of the multiple types of physiological measures, the determiner 13 determines whether the physiological measure exceeds the threshold value corresponding to that physiological measure in each of the evaluation sub-periods. The score deriver 14 derives the number of physiological measures determined to exceed the threshold value in each of the evaluation sub-periods as the sub-score.

As an example, in a first evaluation sub-period, the RRI, CvRR, and RSPI are each acquired as physiological measures, and each of these three types of physiological measures is determined to exceed the threshold value corresponding to that physiological measure. In this case, since there are three physiological measures determined to exceed the threshold value, the score deriver 14 derives a sub-score of 3.As another example, in a second evaluation sub-period, one of the three types of physiological measures is determined to exceed the threshold value corresponding to that physiological measure. In this case, since there is one physiological measure determined to exceed the threshold value, the score deriver 14 derives a sub-score of 1. As another example, in a third evaluation sub-period, none of the three types of physiological measures is determined to exceed the threshold value corresponding to that physiological measure. In this case, since there are zero physiological measures determined to exceed the threshold value, the score deriver 14 derives a sub-score of 0.

In other words, for each of the three types of physiological measures in an evaluation sub-period, the score deriver 14 assigns 1 to a physiological measure that exceeds the threshold value and 0 to a physiological measure that does not exceed the threshold value. The score deriver 14 then adds up the numbers assigned to each of the three types of physiological measures to calculate the sub-score in that evaluation sub-period.

Consequently, when there are three types of physiological measures, the sub-score in the evaluation sub-period is indicated in the four steps of 0, 1, 2, and 3. Note that if a sub-score of 0 is not included among the steps, the sub-score is indicated in the three steps of 1, 2, and 3. The controller 16 generates a graph indicating the change in meditative state by arranging the sub-scores derived for each of the evaluation sub-periods in chronological order, and displays the graph in the meditation change graph area a4.

In this way, the terminal device 10 in the present embodiment executes the acquisition processing, determination processing, and derivation processing described above in each of multiple periods. Each of the periods is the evaluation sub-period described above. Additionally, the terminal device 10 arranges and displays the sub-scores, that is, the scores derived in each of the periods, in chronological order. With this arrangement, since a score related to the meditative state that changes from moment to moment is fed back, the person P experiencing meditation can grasp their own meditative state in detail and be further motivated to meditate. The person experiencing meditation can compare their own bodily sensations to the sub-scores arranged in chronological order.

The terminal device 10 in the present embodiment derives an overall score according to the sub-scores derived in each of the periods, and outputs the overall score. That is to say, the overall score is the meditation score described above, and is displayed in the meditation score area a1. With this arrangement, since the meditation score in the meditation evaluation period including all of the evaluation sub-periods is derived and displayed, the meditative score can be scored for the entire meditation evaluation period. As a result, the person P experiencing meditation can easily get an overall grasp of how well they meditated. Note that the score deriver 14 may calculate the overall score by adding up the sub-scores in each of the evaluation sub-periods, and the controller 16 may display the overall score as the meditation score described above in the meditation score area a1.

The terminal device 10 in the present embodiment derives the sub-score expressed in n or (n+1) steps depending on whether each of the n types of physiological measures exceeds the threshold value corresponding to that type of physiological measure. With this arrangement, the sub-score is expressed in a number of steps according to the number of types (namely, n types) of physiological measures that are acquired, and thus the greater the number of physiological measures is, the more detailed is the sub-score that can be fed back to the person P experiencing meditation.

The evaluation sub-period is 90 seconds long, for example. Each of the evaluation sub-periods partially overlaps with the preceding evaluation sub-period. For example, an evaluation sub-period and the preceding evaluation sub-period are 10 seconds apart and overlap by 80 seconds. Therefore, the sub-score is derived every 10 seconds. Note that the evaluation sub-period is also referred to as the time window.

That is to say, the heart rate variability should be calculated using multiple RRIs in a given time window. Therefore, the measure acquirer 12 calculates the CvRR using multiple RRIs in a 90 second time window, for example, shifts the time window by 10 seconds, for example, and calculates the CvRR using multiple RRIs in the next 90 second time window. The measure acquirer 12 calculates the mean of the RRI and the mean of the RSPI for the time window. With this arrangement, the mean of the RRI, the CvRR, and the mean of the RSPI are calculated for a single 90 second time window. Thereafter, by shifting the 90 second time window by 10 seconds and performing similar calculations, the mean of the RRI, the CvRR, and the mean of the RSPI are calculated every 10 seconds. For each of these time windows (that is, evaluation sub-periods), the determiner 13 determines whether the mean of the RRI, the CvRR, and the mean of the RSPI calculated for that time window exceed the threshold value corresponding to each. The score deriver 14 then derives the sub-score in three or four steps as described above according to the determination result. With this arrangement, bars each having a length expressed in three or four steps are arranged every 10 seconds on the graph in the meditation change graph area a4.

In this way, in the present embodiment, each of the evaluation sub-periods (that is, time windows) partially overlaps with the preceding evaluation sub-period. With this arrangement, even if a long time is required to acquire the physiological measures in each of the evaluation sub-periods, the sub-scores can be expressed at short intervals because each of the evaluation sub-periods partially overlaps with the preceding period. As a result, the time resolution of the sub-scores can be increased, and detailed change in the sub-scores over time can be fed back to the person P experiencing meditation.

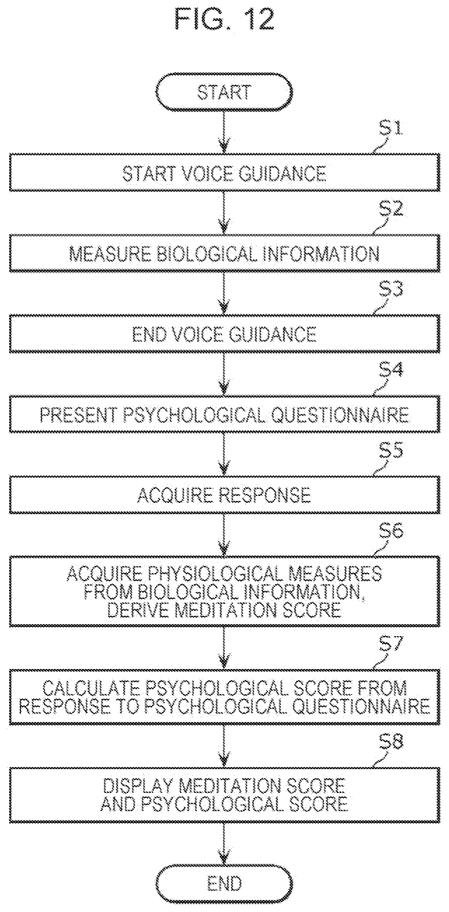

FIG. 12 is a flowchart illustrating an example of processing operations by the terminal device 10.

First, the controller 16 of the terminal device 10 starts the output of voice guidance from the sound outputter 18 (step S1). Note that the output of voice guidance may be started at the start point of the rest period described above, for example. Thereafter, the measure acquirer 12 measures an electrocardio waveform of the person P as biological information on the basis of a sensing signal transmitted from the sensor device 20 (step S2). The controller 16 then ends the output of voice guidance from the sound outputter 18 (step S3). Note that the output of voice guidance may be ended at the end point of the meditation period described above, for example. Next, the questionnaire processor 15 displays the psychological questionnaire on the display 17 (step S4), and acquires a response to each question of the psychological questionnaire (step S5).

Next, the measure acquirer 12 acquires multiple types of physiological measures from the biological information measured in step S2. For each of the multiple types of physiological measures, the determiner 13 determines whether the physiological measure exceeds the threshold value corresponding to that physiological measure. Thereafter, the score deriver 14 derives the meditation score of the person P on the basis of the result of the determination by the determiner 13 (step S6). Furthermore, the questionnaire processor 15 calculates the psychological score of the person P on the basis of the response to each question acquired in step S5 (step S7).

Thereafter, the controller 16 displays the meditation score of the person P derived in step S6 and the psychological score calculated in step S7 on the display 17.

Note that in step S6, the sub-score in each evaluation sub-period may also be calculated. In step S6, these sub-scores may be displayed in addition to the meditation score, and each individual score may also be displayed. The order of the processing in steps S4 to S7 is not limited to the example in FIG. 12. For example, the processing in step S6 may be performed before step S4 or S5, and the processing in step S7 may be performed before step S6.

As above, in the information processing method according to the present embodiment, n types of physiological measures are acquired, it is determined whether each of the acquired n types of physiological measures exceeds a threshold value corresponding to that type of physiological measure, and a score related to the meditative state of a person is derived on the basis of the determination result. With this arrangement, the score can be fed back to the person. As a result, it is possible to motivate a person to meditate and keep that person meditating repeatedly in an ongoing way.

In the information processing method according to the present embodiment, response data indicating a response by a person to a psychological questionnaire is acquired after the person was in a state for meditation, and psychological information indicating a change in the psychological state of the person is generated on the basis of the response data and presented to the person. With this arrangement, it is possible to provide feedback on the psychological effects of meditation. As a result, it is possible to further motivate a person to meditate and keep that person meditating repeatedly in an ongoing way.

The above describes an information processing method and terminal device according to one or more aspects of the present disclosure on the basis of the embodiment, but the present disclosure is not limited to the embodiment. Embodiments obtained by applying various modifications that may occur to a person skilled in the art to the above embodiment may also be included in the scope of the present disclosure insofar as such embodiments do not depart from the gist of the present disclosure.

For example, the terminal device 10 in the above embodiment is provided with various components as illustrated in FIG. 3, but some or all of these components do not have to be provided. In this case, the component(s) not provided in the terminal device 10 may be provided in a server that can communicate with the terminal device 10. For example, a component such as the score deriver 14 may be provided in a server. By communicating with the server, the terminal device 10 performs processing operations similar to the above embodiment.

The terminal device 10 in the above embodiment measures an electrocardio waveform of the person P, but may also measure a pulse wave instead of the electrocardio waveform. In this case, the terminal device 10 acquires the physiological measures described above from the pulse wave. Furthermore, in this case, the sensor device 20 is not limited to be applied to the chest and may also be applied to the fingers or wrists. The terminal device 10 may also be provided with a camera for measuring a pulse wave. The person P places their finger against the camera, and the camera captures an image of the finger. The terminal device 10 measures the pulse wave on the basis of changes in the skin chromaticity of the finger captured in the image. The camera may also capture an image of the face of the person P. In this case, the terminal device 10 measures the pulse wave on the basis of changes in the skin chromaticity of the face.

The terminal device 10 in the above embodiment acquires the respiratory cycle from the electrocardio waveform, but may also acquire the respiratory cycle from the tension imparted to a belt wrapped around the chest or abdomen. For example, the belt transmits to the terminal device 10 a sensing signal indicating the tension imparted to the belt. The terminal device 10 receives the sensing signal from the belt and acquires the respiratory cycle on the basis of the tension indicated by the sensing signal. The respiratory cycle may also be acquired on the basis of a phenomenon other than the electrocardio waveform or the above tension.

In the above embodiment, the terminal device 10 derives and displays the meditation score, the sub-scores, and the psychological score, but may also derive and display only the meditation score or the sub-scores, or derive and display only the psychological score. The psychological score may also be displayed as a numerical value instead of, or in addition to, an arrow, and may also be displayed as a bar of a length corresponding to the numerical value.

In the above embodiment, the terminal device 10 guides the person P into meditation, but the type of meditation may be meditation for concentration (that is, concentration meditation) or meditation for awareness (that is, insight meditation). The meditation is not limited to these types and may be any of any type, such as mindfulness, seated Zen meditation, and yoga.

In the above embodiment, six types, three types, or the like of physiological measures are acquired, but a single type of physiological measure may be acquired, and a score may be derived on the basis of the single type of physiological measure.

In the above embodiment, the heartbeat interval is used as a physiological measure in the determination processing, but a value such as the instantaneous heart rate may also be used instead of the heartbeat interval. In this case, the determiner 13 does not determine whether the instantaneous heart rate exceeds a threshold value, but rather determines whether the instantaneous heart rate falls below a threshold value, and the score deriver 14 derives a score on the basis of the determination result. Similarly, in the above embodiment, the CvRR, LF, HF, and LF+HF are used as the heart rate variability in the determination processing, but another physiological measure indicating heart rate variability may also be used instead of, or in addition to, the above.

Note that in the above embodiment, each component may be configured by dedicated hardware or achieved by executing a software program suited to each component. Each component may be achieved as a result of a program execution unit such as a CPU or a processor reading out and executing a software program recorded on a recording medium such as a hard disk or semiconductor memory. The software program for achieving the terminal device and the like of the above embodiment causes a computer to execute each of the steps included in the flowchart illustrated in FIG. 12.

Note that cases like the following are also included in the present disclosure.
(1) At least one device above is specifically a computer system formed from, among other things, a microprocessor, read-only memory (ROM), random access memory (RAM), a hard disk unit, a display unit, a keyboard, and a mouse. A computer program is stored in the RAM or the hard disk unit. The at least one device above achieves the functions thereof as a result of the microprocessor operating in accordance with the computer program. The computer program herein contains a combination of instruction codes that indicate commands to the computer to achieve certain functions.
(2) Some or all of the components forming the at least one device above may also be configured as a single system large-scale integration (LSI) chip. A system LSI chip is an advanced multi-function LSI chip fabricated by integrating multiple components onto a single chip, and specifically is a computer system including a microprocessor, ROM, RAM, and the like. A computer program is stored in the RAM. The microprocessor operates in accordance with the computer program and thereby achieves the functions of the system LSI chip.
(3) Some or all of the components forming the at least one device above may also be configured as an IC card or standalone module that can be removably inserted into the device. The IC card or module is a computer system formed from a microprocessor, ROM, RAM, and the like. The IC card or module may also include the advanced multi-function LSI chip above. The microprocessor operates in accordance with the computer program and thereby achieves the functions of the IC card or module. The IC card or module may also be tamper-resistant.
(4) The present disclosure may also be the methods indicated above. In addition, these methods may be a computer program to be achieved by a computer, or a digital signal containing the computer program.

The present disclosure may also be achieved by recording the computer program or the digital signal onto a computer-readable recording medium, such as a flexible disk, hard disk, Compact Disc ROM (CD-ROM), DVD, DVD-ROM, DVD-RAM, Blu-ray® Disc (BD), or semiconductor memory, for example. The present disclosure may also be the digital signal recorded on these recording media.

The present disclosure may also be achieved by transmitting the computer program or digital signal over a telecommunications line, a wired or wireless communication channel, a network such as the Internet, or a data broadcast.

The present disclosure may also be carried out by another independent computer system by recording and transporting the program or digital signal on a recording medium, or by transferring the program or digital signal over a network.

The present disclosure is usable in a device or system used to experience meditation, for example.

What is claimed is:

1. An information processing method to be performed by a computer, the information processing method comprising:
executing acquisition processing that acquires n types (where n is an integer equal to or greater than 1) of physiological measures of a person in a state for meditation;
executing determination processing that determines whether each of the acquired n types of physiological measures exceeds a threshold value corresponding to that type of physiological measure;
executing derivation processing that derives a score related to a meditative state of the person on the basis of the result of the determination processing; and
outputting the derived score.

2. The information processing method according to claim 1, wherein
one of the n types of physiological measures is the heartbeat interval of the person, and
provided that α denotes the heartbeat interval of the person in a resting state and x denotes the threshold value corresponding to the heartbeat interval,
the threshold value x satisfies 1.0α≤x≤1.2α.

3. The information processing method according to claim 1, wherein
one of the n types of physiological measures is the heart rate variability of the person, and
provided that β denotes the heart rate variability of the person in a resting state and y denotes the threshold value corresponding to the heart rate variability,
the threshold value γ satisfies 1.0β≤y≤2.1β.

4. The information processing method according to claim 1, wherein
one of the n types of physiological measures is the respiratory cycle of the person, and
provided that γ denotes the respiratory cycle of the person in a resting state and z denotes the threshold value corresponding to the respiratory cycle,
the threshold value z satisfies 1.0γ≤z≤1.3γ.

5. The information processing method according to claim 1, wherein
the acquisition processing, the determination processing, and the derivation processing are executed in each of multiple periods, and
in the outputting of the score, the scores derived in each of the periods are arranged and displayed in chronological order.

6. The information processing method according to claim 5, wherein
in the derivation processing, an overall score is derived according to the scores derived in each of the periods, and
the information processing method further includes outputting the overall score.

7. The information processing method according to claim 5, wherein in the derivation processing, the score expressed in n or (n+1) steps is derived depending on whether each of the n types of physiological measures exceeds the threshold value corresponding to that type of physiological measure.

8. The information processing method according to claim 5, wherein each of the periods partially overlaps with the preceding period.

9. The information processing method according to claim 1, further comprising:
acquiring, after the person was in a state for meditation, response data indicating the result of a response by the person to a questionnaire related to psychological state;
generating, on the basis of the acquired response data, psychological information indicating a change in the psychological state of the person after the person was in the state for meditation; and
presenting the generated psychological information to the person.

10. The information processing method according to claim 9, wherein
the questionnaire is a questionnaire asking about the degree to which the person is relaxed, and
the psychological information indicates a change in the degree of relaxation.

11. The information processing method according to claim 10, wherein the questionnaire is a questionnaire asking about the degree of at least one psychological state of the person from among fatigue, nervousness, and exhilaration as the degree of relaxation, and
the psychological information indicates a change in the degree of psychological state for each of the at least one psychological state.

12. An information processing method to be performed by a computer, the information processing method comprising:
acquiring, after a person was in a state for meditation, response data indicating the result of a response by the person to a questionnaire related to psychological state;
generating, on the basis of the acquired response data, psychological information indicating a change in the psychological state of the person after the person was in the state for meditation; and
presenting the generated psychological information to the person.

13. The information processing method according to claim 12, wherein
the questionnaire is a questionnaire asking about the degree to which the person is relaxed, and
the psychological information indicates a change in the degree of relaxation.

14. The information processing method according to claim 13, wherein
the questionnaire is a questionnaire asking about the degree of at least one psychological state of the person from among fatigue, nervousness, and exhilaration as the degree of relaxation, and
the psychological information indicates a change in the degree of psychological state for each of the at least one psychological state.

15. An information processing method to be performed by a computer, the information processing method comprising:
executing acquisition processing that acquires n types (where n is an integer equal to or greater than 1) of physiological measures of a person in a state for meditation;
executing derivation processing that derives a score related to a meditative state of the person in each of multiple periods on the basis of each of the acquired n types of physiological measures; and
outputting the scores derived in each of the periods such that the scores are arranged and displayed in chronological order.

16. The information processing method according to claim 15, wherein
in the derivation processing, an overall score is derived according to the scores derived in each of the periods, and
the information processing method further includes outputting the overall score.

17. The information processing method according to claim 16, wherein the overall score is the sum of the scores derived in each of the periods.

18. The information processing method according to claim 15, wherein in the derivation processing, the score expressed in n or (n+1) steps is derived.

19. A terminal device comprising:
a physiological measure acquirer that acquires n types (where n is an integer equal to or greater than 1) of physiological measures of a person in a state for meditation;
a determiner that determines whether each of the acquired n types of physiological measures exceeds a threshold value corresponding to that type of physiological measure;
a score deriver that derives a score related to a meditative state of the person on the basis of the result of the determination by the determiner; and
an outputter that outputs the derived score.

20. A terminal device comprising:
a questionnaire processor that acquires, after a person was in a state for meditation, response data indicating the result of a response by the person to a questionnaire related to psychological state and generates, on the basis of the acquired response data, psychological information indicating a change in the psychological state of the person after the person was in the state for meditation; and
an outputter that presents the generated psychological information to the person.

21. A non-transitory computer-readable recording medium storing a program causing a computer to execute processing comprising:
executing acquisition processing that acquires n types (where n is an integer equal to or greater than 1) of physiological measures of a person in a state for meditation;
executing determination processing that determines whether each of the acquired n types of physiological measures exceeds a threshold value corresponding to that type of physiological measure;
executing derivation processing that derives a score related to a meditative state of the person on the basis of the result of the determination processing; and
outputting the derived score.

22. A non-transitory computer-readable recording medium storing a program causing a computer to execute processing comprising:
acquiring, after the person was in a state for meditation, response data indicating the result of a response by the person to a questionnaire related to psychological state;
generating, on the basis of the acquired response data, psychological information indicating a change in the psychological state of the person after the person was in the state for meditation; and
presenting the generated psychological information to the person.

23. An information processing method to be performed by a computer, the information processing method comprising:
acquiring one or more values of one or more physiological measures of a person, the one or more physiological measures corresponding one-to-one to one or more threshold values, the one or more values being measured after an output of voice guidance is started;
determining whether each of the acquired one or more values exceeds a threshold value included among the one or more threshold values;
calculating a score of the person on the basis of the determination; and
outputting the derived score.

24. The information processing method according to claim 23, wherein
the one or more physiological measures include a heartbeat interval of the person,
the one or more threshold values include a first threshold value,
the heartbeat interval corresponding to the first threshold value,
the condition 1.0α≤x≤1.2α is met, where a is the heartbeat interval of the person in a resting state and x is the first threshold value, and
the score is related to a meditative state of the person.
